(19) United States
(12) Patent Application Publication
(10) Pub. No.: US 2020/0069176 A1
(43) Pub. Date: Mar. 5, 2020

(54) System for Approximate Refraction of Patients with a Single, Central Scotoma

(71) Applicant: Ronald Steven Black, Charlotte, NC (US)

(72) Inventor: Ronald Steven Black, Charlotte, NC (US)

(21) Appl. No.: 16/399,890

(22) Filed: Apr. 30, 2019

Related U.S. Application Data
(60) Provisional application No. 62/665,431, filed on May 1, 2018.

(51) Int. Cl. A61B 3/103; A61B 3/117; G16H 10/60; G06F 17/15; G06F 17/12

(52) U.S. Cl. CPC A61B 3/103; A61B 3/1176; G06F 17/12; G06F 17/15; G16H 10/60

(57) ABSTRACT

This patent describes a machine that allows patients with a single, central scotoma to receive an acceptable but sub-optimal refraction for the badThanks. [symbol] eye based on previous refraction values, current refraction of the Good Eye, cataract characteristics, and subjective judgements of whether the patient can identify optotypes after incremental changes in refraction.

### DEFINITIONS

Aniseikonia: A defect of binocular vision in which the two retinal images of an object differ in size.
Automated refractor: A machine that uses laser or other means to scan the eye automatically and determine the refractive error and preferred lens prescription.
Bad Eye: For purposes of this report, the patient's eye with a central scotoma.
Cycloplegic: a compound inducing paralysis of the ciliary muscle of the eye.
Fovea: A small depression in the center of the macula that contains only cones and constitutes the area of maximum visual acuity and color discrimination.
Good Eye: For purposes of this report, the patient's eye without a central scotoma.
Macula: A small yellowish area lying slightly lateral to the center of the retina that is made up mostly of cones, plays a key role in visual acuity, and has the fovea at its center.
Optotype: Figures or letters of different sizes used in testing the acuity of vision.
Phoropter: An instrument used to determine the corrective eyeglass lenses needed by a person.
Refraction: The act or technique of determining the ocular refraction and identifying abnormalities as a basis for the prescription of corrective lenses.
Scotoma: A spot in the visual field in which vision is absent or deficient.

### Acronyms

The following acronyms are used in this document:

| Acronym | Expansion |
|---|---|
| CPC | Cooperative Patent Classification |
| LOCS | Lens Opacities Classification System |
| logMAR | Logarithmic Minimum Angle of Resolution |
| USPTO | United States Patent and Trademark Office |

### PROBLEM STATEMENT

Standard refraction techniques used by vision care providers place a screen with optotypes at 20 feet (6 meters) from the patient and use a phoropter after administration of a cycloplegic compound to develop the optimal refraction for greatest acuity in the foveal region. The screen is small and does not extend significantly to either side of the foveal field of vision. Patients with a central scotoma in one eye are not amenable to these standard techniques in that eye, because the foveal region of their retina is degraded or non-functional. Automated refractors give an approximate refraction, but the result is far from optimal. Patients with a central scotoma thus experience a degradation of peripheral acuity over time in the affected eye, because they are unable to respond to standard refraction techniques as the eye physiology changes. Amblyopia can occur in the Bad Eye, and as one lens prescription changes over time but the other does not the different refractions can also cause aniseikonia.

Low vision care specialists can use non-standard techniques for refraction of patients with degraded foveal acuity. Patients are generally referred to low vision care specialists only after acuity has degraded in both eyes. There are two reasons: It is assumed that the “good” eye will make up for the low vision in the other eye, and the paucity of low vision care specialists relative to patients with low vision is also a component in the calculus of referral. Low vision care specialists are in high demand, particularly given the growing incidence of age-related macular degeneration. General ophthalmologists and optometrists refer only patients with needs in both eyes, because the pool of patients with a single, central scotoma is much larger than the pool of low vision care specialists can accommodate.

The process of determining a refraction for a patient with a central scotoma in one eye is long and frustrating both for the patient and the vision care provider, and is unlikely to achieve a significantly better result than previous refractions. Until loss of vision occurs in both eyes, refractions for the Bad Eye in patients with a central scotoma are generally left at the last accurate value.

### DESCRIPTION OF THE INVENTION

### System Modules

FIG. 1 presents a block diagram of the machine and shows information entered into and produced by each module. The machine consists of the following modules:

1. Estimator Modules

These modules take as input the following values for a set of previous refractions for both eyes. All the values are before the emergence of the central scotoma in the Bad Eye:
a. Date of refraction
b. Left spherical power (in diopters)
c. Left cylindrical power (in diopters)
d. Left cylindrical angle (in degrees)
e. Right spherical power (in diopters)
f. Right cylindrical power (in diopters)
g. Right cylindrical angle (in degrees)

The number of refractions may vary, but preliminary results indicate that values from seven previous refractions are sufficient. The Estimator Modules then use three methods of data correlation to determine the relationships between the left and right spherical powers, left and right cylindrical powers, and left and right cylindrical angles. The methods are linear correlation; second order polynomial correlation; and first-order linear difference equation modeling. The coefficient values for each of the modeling methods and the confidence intervals are transmitted to the Test and Results Comparator Module.

2. Test and Results Comparator Module

This module takes as input the following values for a set of previous refractions for both eyes. All the values are before the emergence of the central scotoma in the weak eye but after the refractions used in the Refraction Correlation Module:
a. Date of refraction
b. Left spherical power (in diopters)
c. Left cylindrical power (in diopters)
d. Left cylindrical angle (in degrees)
e. Right spherical power (in diopters)
f. Right cylindrical power (in diopters)
g. Right cylindrical angle (in degrees)

The number of refractions may vary, but preliminary results indicate that values from two previous refractions are sufficient. This module also receives the correlation coefficients and confidence intervals from each of the three Refraction Correlation Modules.

Because linear modeling is the simplest and most robust given the relatively small amount of data, if the correlation coefficient of the linear model is 0.85 or greater, the linear model is used. Otherwise, using the correlation coefficients from each of the Estimator Modules, the Test and Results Comparator Module calculates an estimated refraction for the Bad Eye based on the refraction values for the Good Eye in each of the refraction histories. The module then compares the difference between the estimated refraction and the actual refraction for each of the histories. The correlation method with the most accurate prediction as measured by difference from the actual refraction is chosen as the refraction estimator.

3. Standard Refraction Estimate Module

This module takes as input the following data from the current refraction of the Good Eye:
a. Date of refraction
b. Spherical power (in diopters)
c. Cylindrical power (in diopters)
d. Cylindrical angle (in degrees)
Using the correlation method and coefficients determined by the Test and Results Comparator Module, this module estimates a refraction for the weak eye based on current refraction values for the Good Eye.

4. Cataract Refraction Estimate Module

If there is a cataract in either eye, this module takes as input the following data:
a. Date of refraction
b. Good Eye spherical power (in diopters)
c. Good Eye cylinder power (in diopters)
d. Good Eye cylinder angle (in degrees)
e. Date of first cataract diagnosis for the Good Eye
f. LOCS measurement for the Good eye
g. Date of first cataract diagnosis for the Bad Eye
h. LOCS measurement for the Bad Eye
i. For each refraction since the first cataract diagnosis in either eye:
   i. Date of refraction
   ii. LOCS III measurement for the Good Eye
   iii. LOCS III measurement for the Bad Eye
   iv. Type of cataract in the Good Eye
   v. Type of cataract in the Bad Eye
   vi. Good Eye refraction spherical power (in diopters)
   vii. Good Eye refraction cylindrical power (in diopters)
   viii. Good Eye refraction cylindrical angle (in degrees)
   ix. Bad Eye actual or estimated refraction spherical power (in diopters)
   x. Bad Eye actual or estimated refraction cylindrical power (in diopters)
   xi. Bad Eye actual or estimated refraction cylindrical angle (in degrees)

The Cataract Refraction Estimate Module then calculates the estimated refraction for the Bad Eye based on the historical response of the eyes to the presence of cataracts and the rate of progression of the cataracts over time as determined by LOCS III. Cataract type may be used to fine tune the estimate, but preliminary results indicate that type is not a heavily influencing factor. Only the spherical power of the refraction for the Bad Eye is affected by the presence of cataracts using this machine. Basing only the spherical power on cataract characteristics simplifies the machine's operation and has little if any effect on the patient's acuity in the Bad Eye.

The Estimator and Test and Results Comparator Modules are only used in the initial patient encounter. The correlation method and correlation coefficient values determined by these modules are used to estimate the refraction for the eye with a central scotoma in subsequent patient visits.

### Operation

When the patient first has difficulties in getting an accurate refraction in the Bad Eye, the vision care provider takes the following steps:
1. Using records of past refractions, enter the prescribed values into the Estimator Modules through a data entry screen.
2. Perform a refraction on the patient's Good Eye.
3. Enter the following information into the machine:
   a. Current refraction values for the Good Eye
   b. Cataract type and LOCS III measurement for the Good Eye, if applicable
   c. Date of initial diagnosis of cataract in the Good Eye, if applicable
   d. Cataract type and LOCS III measurement for the Bad Eye, if applicable
   e. Date of initial diagnosis of cataract in the Bad Eye, if applicable
4. Enter the corrected estimate into a phoropter, and ask the patient to compare the current refraction of the weak eye with the last recorded refraction.
5. If the patient discerns an improvement in acuity, prescribe the estimated refraction.

### Implementation

The modules described above can be implemented using software, non-programmable digital electronic components, analog electronic components, or a combination of the three. Software has been used to simulate the implementation in developing this patent specification, and it is planned to implement the machine using software. It is not, however, required that that software be used. The method of implementation is not germane to the nature of the innovation inherent in this device.

As stated above, FIG. 1 is a block diagram showing the structure of the machine and information passing into and out of each module. FIG. 2 is a flowchart of the Estimator, Test, and Results Comparator Modules. FIGS. 3 through 8 show progressively more detailed flowcharts for the planned implementation of the refraction procedure modules.

### BRIEF DESCRIPTIONS OF THE DRAWINGS

FIG. 1 is a system block diagram showing major modules and data flows.

FIG. 2 is a flowchart of the Estimator, Test, and Results Comparator Modules

FIG. 3 is a flowchart of the procedure for Bad Eye refraction.

FIG. 4 is a flowchart of the procedure for Bad Eye refraction with a cataract in either eye.

FIG. 5 is a flowchart of Cataract Refraction Procedure 1 (Cataract in Bad Eye Only).

FIG. 6 is a flowchart of Cataract Refraction Procedure 2 (Cataract in Good Eye Only).

FIG. 7 is a flowchart of Cataract Refraction Procedure 3 (Cataracts in Both Eyes).

FIG. 8 is a flowchart of Cataract Refraction Procedure 3a (Cataracts in Both Eyes).

### DETAILED DESCRIPTIONS OF THE DRAWINGS

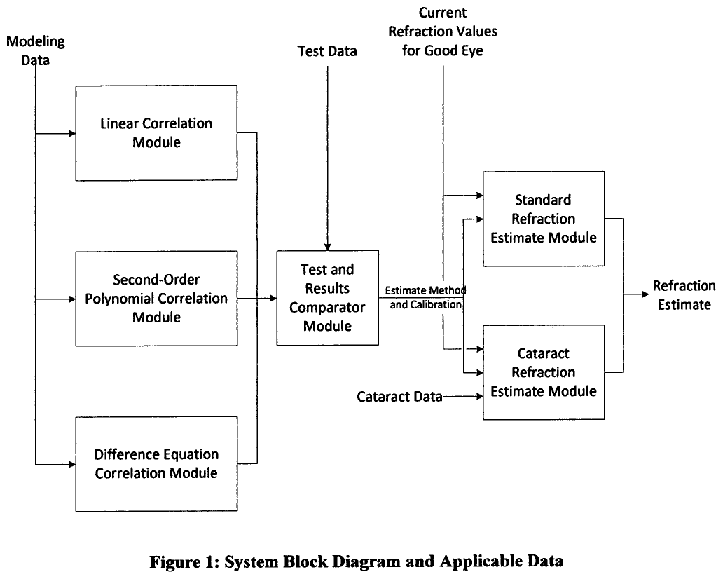

### FIG. 1: Estimator Modules and Data

FIG. 1 is a system block diagram showing major modules and data flows. There are six modules:

1. Linear Correlation Module

2. Second-Order Polynomial Correlation Module

3. Ordinary Difference Equation Correlation Module

4. Test and Results Comparator Module

5. Standard Refraction Estimate Module

6. Cataract Refraction Estimate Module

The patient's refraction history from before the emergence of the scotoma is entered using an input device such as a keyboard and is transferred to the three correlation modules. Refractions are measured and entered in diopters. The Linear Correlation Module uses a subset of the data to determine the linear correlation between the refractions of the left and right eyes over time. It also calculates the correlation coefficient, R², between the two refraction histories, and the 80% confidence intervals of the estimate terms.

If the Second-Order Polynomial Correlation Module is activated, it uses the same subset of the data to determine the correlation between the refractions of the left and right over time using both the refractions and the squares of the refractions. It also calculates R² and the 80% confidence intervals of the estimate terms. If the Ordinary Difference Equation Correlation Module is activated, it uses the same subset of the data for the eye with the scotoma to measure the correlation between the refraction of that eye and the previous refraction of the same eye. This module also calculates R² and the 80% confidence intervals of the estimate terms. After calculating the estimate terms and R², the information is transferred to the Test and Results Comparator Module along with the refraction data not used in calculating the estimates.

The Test and Results Comparator Module uses the estimate results it is given to predict the refractions for the eye with the scotoma using the reserved data not used in determining the estimate. It calculates the errors between the predicted and actual refractions for the eye with the scotoma. If the Second-Order Polynomial Correlation and Ordinary Difference Equation Correlation Modules were not activated, but the errors using the reserved data are not acceptable, the Second-Order Polynomial and Ordinary Difference Equation Correlation Modules are used to determine those estimate terms. Then using each of the three estimate methods, the module calculates predicted values for the reserved data set. It then compares those three error sets and determines the method to be used for predicting future patient refractions. Preference is given to the linear correlation method, because of its simplicity and robustness. The Test and Results Comparator Module then sends the estimate terms and preferred estimate method to the Standard and Cataract Refraction Modules.

In using the system to predict a new refraction for a patient under examination, the vision care provider enters the current refraction of the eye without the scotoma, and whether there is a cataract in either eye. If the patient has no cataract, the Standard Refraction Estimate Module will determine the refraction for the eye with the scotoma. If there is a cataract in either eye, the refraction history of the eye(s) with the cataract(s) since the emergence of the cataract(s) will be entered, and the LOCS III evaluations of the cataract(s) at each examination since its (their) emergence. The Cataract Refraction Estimate Module will then determine the refraction. The provider then uses the estimated refraction for an acuity check. If there is any improvement with the estimate, the provider can prescribe that refraction.

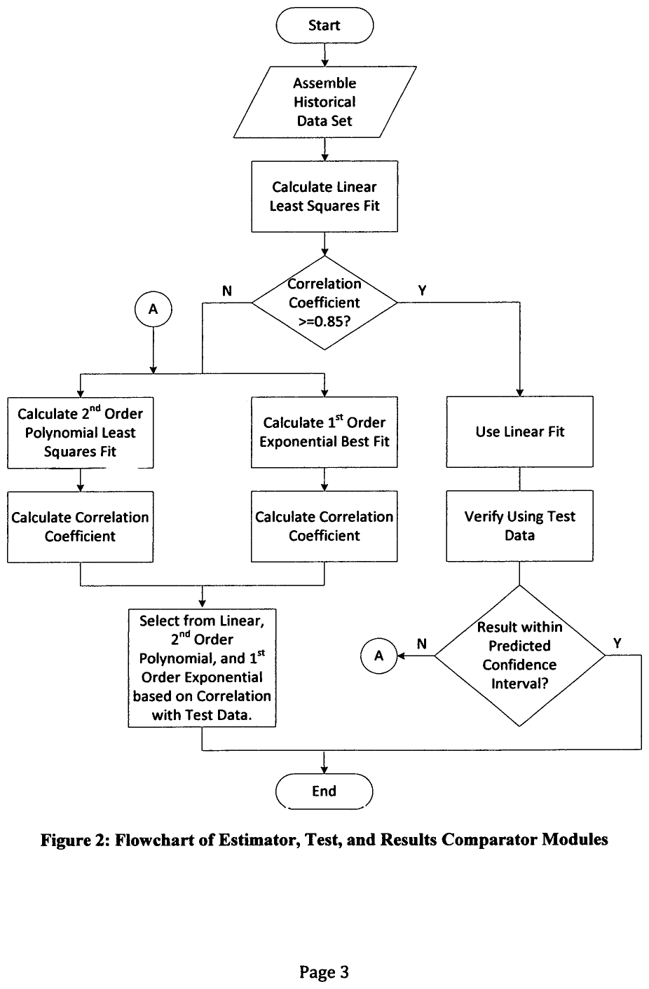

### FIG. 2: Flowchart of the Estimator and the Test and Results Comparator Modules

FIG. 2 is a flowchart showing how data is manipulated and transferred in and between the Estimator Modules and the Test and Results Comparator Module. First, a refraction history for the patient is entered, a set of at least eight refractions performed before the emergence of the scotoma. For each refraction in the history, the following data is entered:

1. Date of refraction

2. Left spherical power (in diopters)

3. Left cylindrical power (in diopters)

4. Left cylindrical angle (in degrees)

5. Right spherical power (in diopters)

6. Right cylindrical power (in diopters)

7. Right cylindrical angle (in degrees)

If the patient has a cataract in either or both eyes, an entry indicating cataract(s) is made in the data, and the following data is entered:

1. Date of refraction

2. Good Eye spherical power (in diopters)

3. Good Eye cylinder power (in diopters)

4. Good Eye cylinder angle (in degrees)

5. Date of first cataract diagnosis for the Good Eye

6. LOCS measurement for the Good Eye

7. Date of first cataract diagnosis for the Bad Eye

8. LOCS measurement for the Bad Eye

9. For each refraction since the first cataract diagnosis in either eye:
a. Date of refraction
b. LOCS III measurement for the Good Eye
c. LOCS III measurement for the Bad Eye
d. Type of cataract in the Good Eye
e. Type of cataract in the Bad Eye
f. Good Eye refraction spherical power (in diopters)
g. Good Eye refraction cylindrical power (in diopters)
h. Good Eye refraction cylindrical angle (in degrees)
i. Bad Eye actual or estimated refraction spherical power (in diopters)
j. Bad eye actual or estimated refraction cylindrical power (in diopters)
k. Bad eye actual or estimated refraction cylindrical angle (in degrees)

The two most recent of the refractions from before the emergence of the scotoma are reserved. Using the others, a linear correlation is calculated for each of the three refraction elements (spherical strength, cylindrical strength, and cylindrical angle). The correlations have the form of y=mx+b, where y is each of the refraction elements of the Bad Eye and x is each of the refraction elements of the Good Eye. These will constitute a set as follows:

mₛ Spherical strength multiplier

bₛ Spherical strength intercept

m_{cs} Cylindrical strength multiplier

b_{cs} Cylindrical strength intercept

m_{ca} Cylindrical angle multiplier

b_{ca} Cylindrical angle intercept

The correlation coefficient, R², and the 80% t values will also be calculated for each of the three elements.

If R² is 0.85 or greater, the following procedure is executed:
1. For each case of the two reserved refractions, identify which refractions were for what is presently the Good Eye and which were for what is presently the Bad Eye.
2. Using the reserved data set, calculate for the Bad Eye predicted values of spherical strength, cylindrical strength, and cylindrical angle, using the m and b coefficients calculated and the refraction data for the Good Eye.
3. Using the reserved data set, calculate the error of the each prediction by comparing it to the actual refractions for the Bad Eye.
4. If all predicted values fall within the 80% t-interval calculated above for each of the refraction elements, the calculated m and b values can be used for future refractions with the scotoma, and the Estimator and Test and Results Comparator Modules' work is completed.

If R² calculated above is less than 0.85, or if any of the test predictions falls outside the 80% t-interval, the following procedure is executed:
1. Perform a second order polynomial least squares fit:
   a. For each element of the historical data set, calculate its square.
   b. Using least squares curve fitting, for each refraction element calculate the m and b values appropriate for the form y=p₁x²+p₂x+p₃. The values will have the form
      p₁s Spherical strength squared multiplier
      p₂s Spherical strength multiplier
      p₃s Spherical strength constant
      p_{1cs} Cylindrical strength squared multiplier
      p_{2cs} Cylindrical strength multiplier
      p_{3cs} Cylindrical strength constant
      p_{1ca} Cylindrical angle squared multiplier
      p_{2ca} Cylindrical angle multiplier
      p_{3ca} Cylindrical angle constant
   c. Using the reserved data set, calculate for the Bad Eye predicted values of spherical strength, cylindrical strength, and cylindrical angle, using the p coefficients calculated and the refraction data for the Good Eye.
   d. Using the reserved data set, calculate the error of the each prediction by comparing it to the actual refractions for the Bad Eye.
2. Calculate a first order difference equation curve fit.
   a. Using only the data for the Bad Eye from the historical data set, for each of the refraction elements, generate a second historical set r′. If there are n elements in the historical set, r′ will have n−1 elements. If rₙ is the most recent value of the refraction element, for each of the n−1 elements of the r′ data set, r′ₖ=rₖ₋₁.
   b. Using least squares curve fitting and the n−1 most recent elements of r and all of r′, calculate the k values appropriate for the form

rₖ=k₁rₖ₋₁+k₂Δt,
where Δt is the time since the last refraction. The values will have the form
   k₁ₛ Spherical strength multiplier
   k₂ₛ Spherical strength rate
   k_{1cs} Cylindrical strength multiplier
   k_{2cs} Cylindrical strength rate
   k_{1ca} Cylindrical angle multiplier
   k_{2ca} Cylindrical angle rate
c. Using the reserved data set, calculate for the Bad Eye predicted values of spherical strength, cylindrical strength, and cylindrical angle, using the m and b coefficients calculated and the refraction data for the Bad Eye.
d. Using the reserved data set, calculate the error of the each prediction by comparing it to the actual refractions for the Bad Eye.
3. If R² for either the polynomial or difference equation curve fitting is 0.10 greater than R² for linear fitting, execute the following procedure:
   a. For spherical strength, cylindrical strength, and cylindrical angle, sum the squares of the errors for each case of linear, polynomial, and difference equation curve fitting.
   b. If the sum of the squares of the errors for one method is 10% or more lower for spherical strength than those of the others, use that method and its calculated coefficients for future refractions with the scotoma, and the Estimator and Test and Results Comparator Modules' work is completed.
   c. Otherwise if the sum of the squares of the errors for one method is 10% or more lower for cylindrical strength than those of the others, use that method and its calculated coefficients for future refractions with the scotoma, and the Estimator and Test and Results Comparator Modules' work is completed.
   d. Otherwise if the sum of the squares of the errors for one method is 10% or more lower for cylindrical angle, use that method and its calculated coefficients for future refractions with the scotoma, and the Estimator and Test and Results Comparator Modules' work is completed.
   e. If the sum of the squares of the errors for no one method is 10% or more lower than the sum of the squared errors for the other methods, the linear method and its calculated m and b values are to be used for future refractions with the scotoma, and the Estimator and Test and Results Comparator Modules' work is completed.
   NOTE: If the chosen method is the second order polynomial method, it is to be used only for refractions that fall between the minimum and maximum values in the original data set. For values that fall outside the ranges of the original data, the linear method and its calculated m and b values are to be used for future refractions with the scotoma.
4. If R² for neither the polynomial nor difference equation curve fitting is 0.10 than R² for linear fitting, the calculated m and b values from the linear curve fitting are to be used for future refractions with the scotoma, and the Estimator and Test and Results Comparator Modules' work is completed.

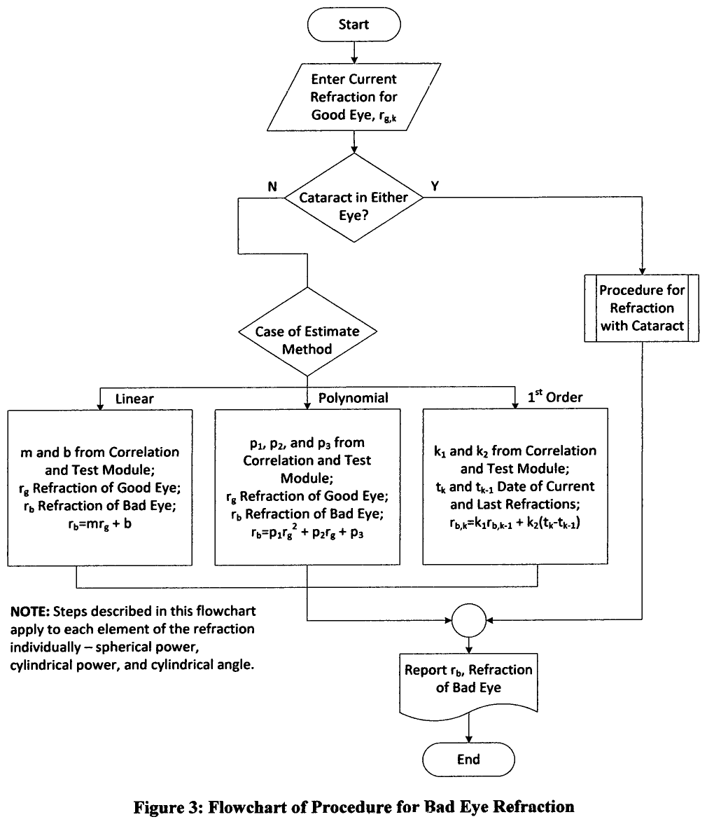

### FIG. 3: Flowchart of Procedure for Bad Eye Refraction

FIG. 3 is a flowchart of the procedure used to calculate the refraction values for the Bad Eye based on the current refraction of the Good Eye. The values calculated for the Bad Eye are spherical power (r_{B,s}), cylindrical power (r_{B,cp}), and cylindrical angle (r_{B,ca}). In this notation, B and G indicate the Bad and Good Eye respectively. The provider enters whether there is a cataract in either eye. If there is a cataract, a separate procedure for refracting an eye when a cataract is present is executed. That procedure is described in subsequent figures.

If there is no cataract, the calculation is performed using a method that depends on the refraction method determined in FIG. 2. The method for each case is described below:

Case 1, Linear
Using the m and b values determined in FIG. 2, calculate refraction values as follows:

r_{B,s}=mₛr_{G,s}bₛ Spherical Power:

r_{B,cs}=m_{cs}r_{G,cs}+b_{cs} Cylindrical Power:

r_{B,cs}=m_{ca}r_{G,ca}+b_{ca} Cylindrical Angle:

Case 2, Polynomial

Using the p values determined in FIG. 2, calculate refraction values as follows:

r_{B,s}=p_{1,s}r²_{G,s}+p_{2,s}r_{G,s}p_{3,s} Spherical Power:

r_{B,cs}=p_{1,cs}r²_{G,cs}+p_{2,cs}r_{G,s}+p_{3,cs} Cylindrical Power:

r_{B,cs}=p_{1,ca}r²_{G,cs}+p_{2,cs}r_{G,ca}+p_{3,cs} Cylindrical Angle:
For each of the calculated r_{B} values, test if it is between the minimum and maximum values used in determining the p coefficients. If it is not, recalculate that value using the linear method as in Case 1.

Case 3, First Order Difference Equation
Using the k values determined in FIG. 2, calculate refraction values as follows:

r_{B,s}=k_{1,s}r_{G,s}+k_{2,s}Δt Spherical Power:

r_{B,cs}=k_{1,cs}+r_{G,cs}k_{2,cs}Δt Cylindrical Power:

r_{B,ca}=k_{1,ca}r_{G,ca}+k_{2,ca}Δt Cylindrical Angle:

Report out to the vision care provider r_{B,s}, r_{B,cs}, and r_{B,ca}. This procedure is now finished.

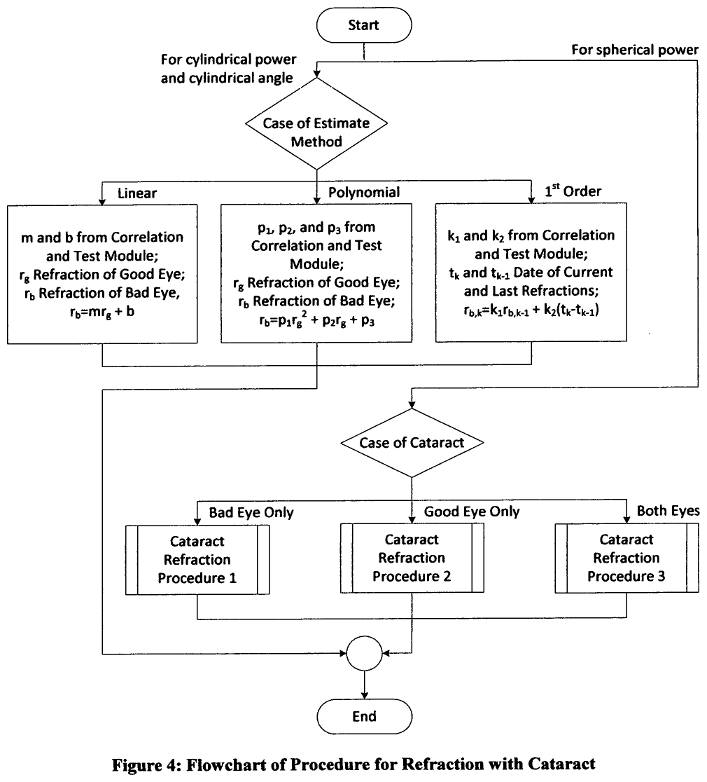

FIG. 4: Flowchart of Procedure for Refraction with Cataract

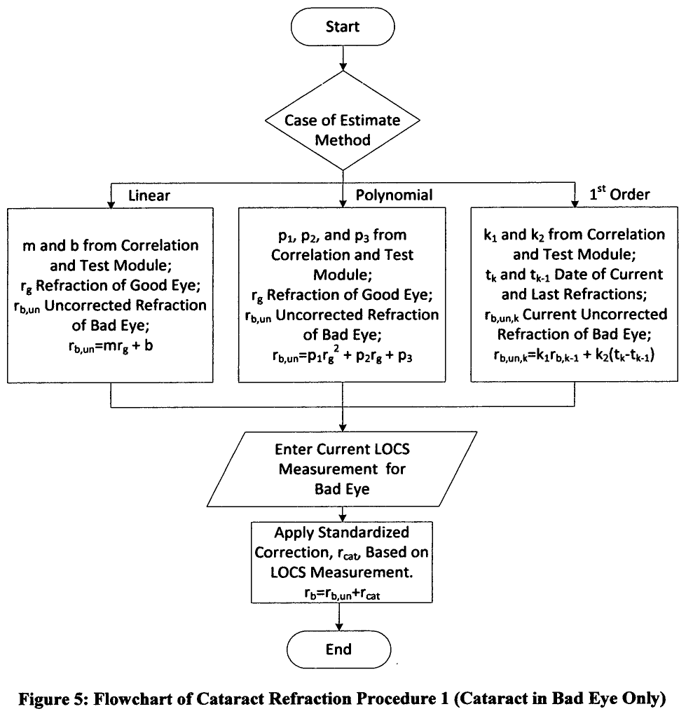

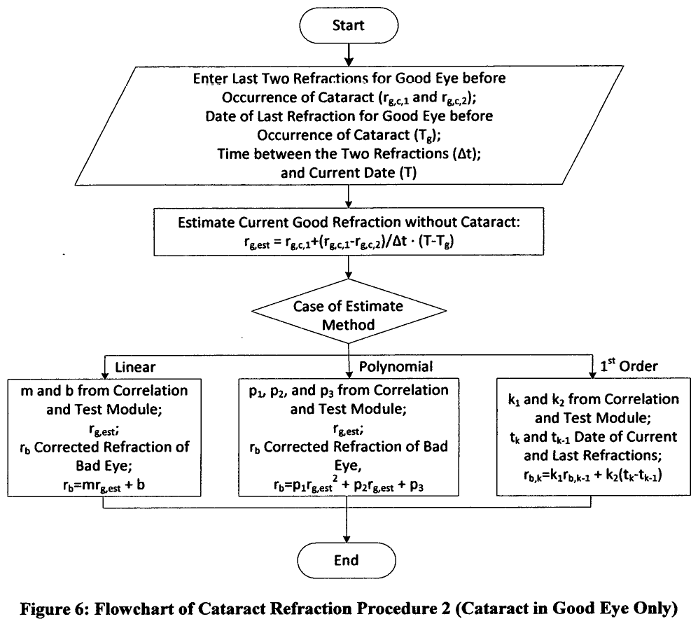

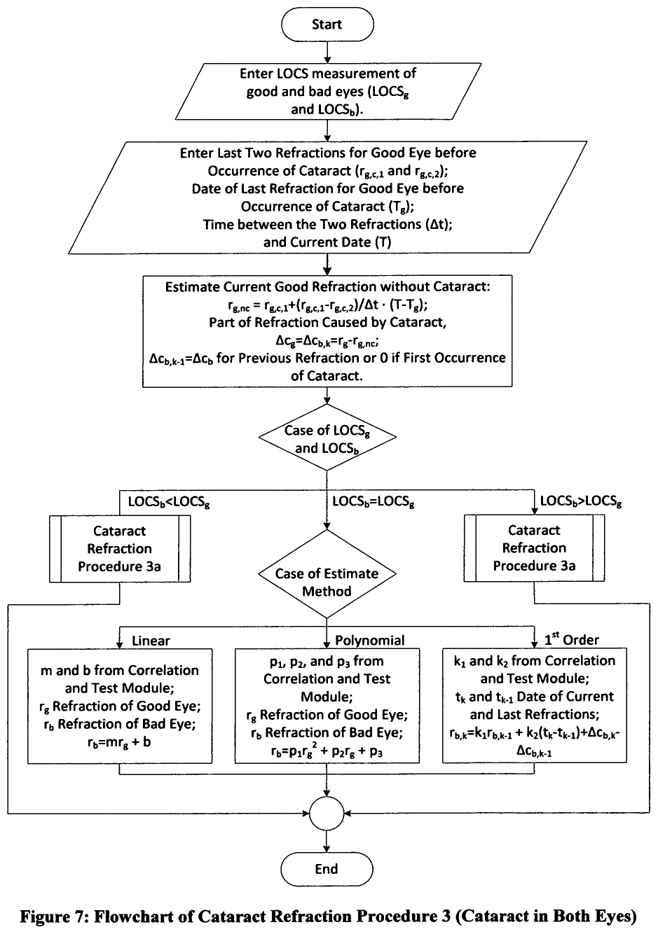

FIG. 4 is a flowchart of the procedure used to calculate the refraction values for the Bad Eye based on the current refraction of the Good Eye given a cataract in one or both eyes. Cylindrical power and cylindrical angle are first calculated using the method determined in FIG. 2 as in the three cases above in FIG. 3. If there is a cataract in the Bad Eye only, execute Cataract Refraction Procedure 1, as shown below in FIG. 5. If there is a cataract in the Good Eye only, execute Cataract Refraction Procedure 2, as shown below in FIG. 6. If there is a cataract in both eyes, execute Cataract Refraction Procedure 3, as shown below in FIG. 7. Report out to the vision care provider r_{B,s}, r_{B,cs}, and r_{B,ca}. This procedure is now finished.

### FIG. 5: Flowchart of Cataract Refraction Procedure 1 (Cataract in Bad Eye Only)

FIG. 5 is a flowchart of the procedure used to calculate the spherical refraction value for the Bad Eye based on the current refraction of the Good Eye given a cataract in the Bad Eye only. First, an uncorrected spherical power calculation is done for the Bad Eye as follows, using the method determined above in FIG. 2:

Case 1, Linear
Using the m and b values determined in FIG. 2, calculate refraction values as follows:

r_{B,s,un}=mₛr_{G,s}+bₛ Spherical Power:

Case 2, Polynomial
Using the p values determined in FIG. 2, calculate refraction values as follows:

r_{B,s,un}=p_{1,s}r²_{G,s}+p_{2,s}r_{G,s}+p_{3,s} Spherical Power:
For each of the calculated r_{B} values, test if it is between the minimum and maximum values used in determining the p coefficients. If it is not, recalculate that value using the linear method as in Case 1.

Case 3, First Order Difference Equation
Using the k values determined in FIG. 2, calculate refraction values as follows:

r_{B,s,un}=k_{1,s}r_{G,s}+k_{2,s}Δt Spherical Power:

Next, the vision care provider enters the cataract type and current LOCS measurement for the Bad Eye. A standardized correction, kₐₜ, is applied to the spherical measurement: r_{B,s}=r_{B,s,un}+r_{B,cat}. This procedure is now finished.

### FIG. 6: Flowchart of Cataract Refraction Procedure 2 (Cataract in Good Eye Only)

FIG. 6 is a flowchart of the procedure used to calculate the spherical refraction value for the Bad Eye based on the current refraction of the Good Eye given a cataract in the Good Eye only. First, enter the last two spherical refraction values for the Good Eye before occurrence of the cataract (r_{G,c,1} and r_{G;c,2}); the date of the last refraction for the Good Eye before the occurrence of the cataract (T_{G}); the time between the two refractions (ΔT); and the current date (T). Next, estimate the spherical refraction of the Good Eye without the cataract (The subscript, “est,” indicates “estimate without cataract,” i.e., the estimated refraction discounting the cataract.):

r_{G,s,est}=r_{G,c,1}+(r_{G,C,1}−r_{G,C,2})/ΔT×(T−T_{G})

Next perform a spherical power calculation for the Bad Eye as follows, using the method determined above in FIG. 2 and r_{G,s,est}:

Case 1, Linear
Using the m and b values determined in FIG. 2, calculate refraction values as follows:

r_{B,s}=mₛr_{G,s,est}+bₛ Spherical Power:

Case 2, Polynomial
Using the p values determined in FIG. 2, calculate refraction values as follows:

r_{B,s}=p_{1,s}r²_{G,s,est}+p_{2,s}r_{G,s,est}+p_{3,s} Spherical Power:
For each of the calculated r_{B} values, test if it is between the minimum and maximum values used in determining the p coefficients. If it is not, recalculate that value using the linear method as in Case 1.

Case 3, First Order Difference Equation
Using the k values determined in FIG. 2, calculate refraction values as follows:

r_{B,s}=k_{1,s}r_{G,s,est}k_{2,s}Δt Spherical Power:

This procedure is now finished.

### FIG. 7: Flowchart of Cataract Refraction Procedure 3 (Cataracts in Both Eyes)

FIG. 7 is a flowchart of the procedure used to calculate the spherical refraction value for the Bad Eye based on the current refraction of the Good Eye given a cataract in the both eyes. First, enter the last two spherical refraction values for the Good Eye before occurrence of the cataract (r_{G,c,1} and r_{G;c,2}); the date of the last refraction for the Good Eye before the occurrence of the cataract (T_{G}); the time between the two refractions (ΔT); and the current date (T). Next, estimate the spherical refraction of the Good Eye without the cataract:

r_{G,s,est}=r_{G,c,1}(r_{G,C,1}−r_{G,C,2})/ΔT×(T−T_{G})

Next estimate the part of the refraction of the Good Eye caused by the cataract:

ΔC_{G}=r_{G,s}−r_{G,s,est}

Next estimate the part of the refraction of the Bad Eye caused by the cataract:

ΔC_{G}=r_{G,s}−r_{G,s,est}

The effect of the cataract in the Bad Eye at the last refraction is used in this calculation. It is indicated by ΔC_{B,k-1}, and is determined by

ΔC_{B,k-1}=ΔC_{B}

for the previous refraction or 0 if this refraction is the first occurrence of a cataract.

Next perform a spherical power calculation for the Bad Eye as follows, based on the relationships of the LOCS measurements of the Good Eye and the Bad Eye:

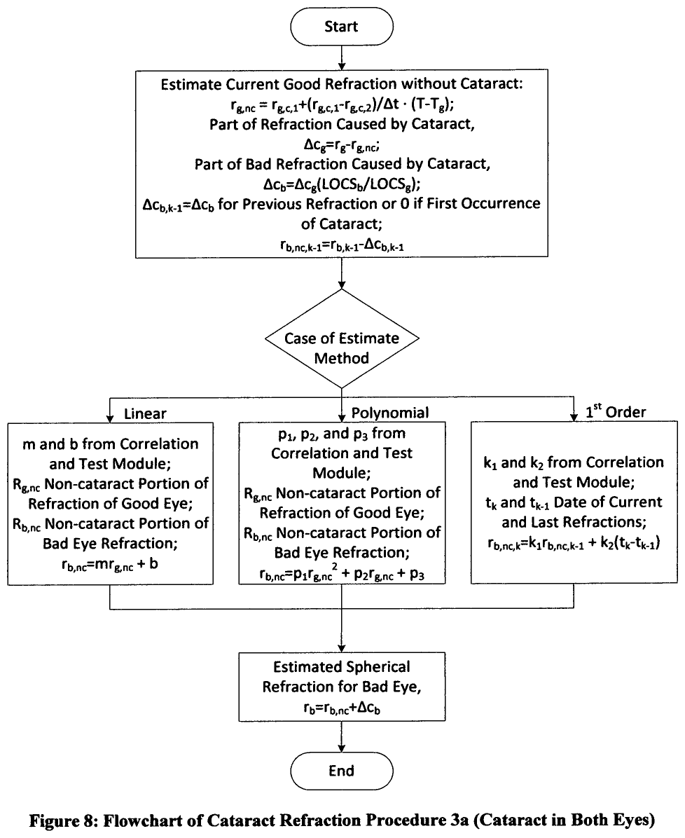

Case 1, LOCS_{B}<LOCS_{G}
Perform Cataract Refraction Procedure 3a as described in FIG. 8. Then this routine is finished.

Case 2, LOCS_{B}=LOCS_{G}
Next perform a spherical power calculation for the Bad Eye as follows, using the method determined above in FIG. 2 and r_{G,s}:
NOTE: r_{G,s} values in these calculations are not corrected values but the measured refractions.

Case a, Linear
Using the m and b values determined in FIG. 2, calculate refraction values as follows:

r_{B,s}=mₛr_{G,s}bₛ Spherical Power:

Case b, Polynomial
Using the p values determined in FIG. 2, calculate refraction values as follows:

r_{B,s}=p_{1,s}r²_{G,s}p_{2,s}r_{G,s}p_{3,s} Spherical Power:
For each of the calculated r_{B} values, test if it is between the minimum and maximum values used in determining the p coefficients. If it is not, recalculate that value using the linear method as in Case a.

Case c, First Order Difference Equation
Using the k values determined in FIG. 2, calculate refraction values as follows:

r_{B,s}=k_{1,s}r_{G,s}+k_{2,s}Δt Spherical Power:
This procedure is now finished.

Case 3, LOCS_{B}>LOCS_{G}
Perform Cataract Refraction Procedure 3a as described in FIG. 8. Then this routine is finished.

### FIG. 8: Flowchart of Cataract Refraction 3a (Cataracts in Both Eyes)

FIG. 8 is a flowchart of the procedure used to calculate the spherical refraction value for the Bad Eye based on the current refraction of the Good Eye given a cataract in the both eyes, when the LOCS classifications of the Bad Eye and the Good Eye are different. Using information from FIG. 7, r_{G,s,est}, ΔC_{G}, and ΔC_{B,k-1} are calculated. The Spherical value of the last refraction discounting the cataract is then calculated:

r_{B,s,nc,k-1}=r_{B,s,k-1}−ΔC_{B,k-1}

Next perform a spherical power calculation for the Bad Eye as follows, using the method determined above in FIG. 2 and r_{G,s}:

Case 1, Linear
Using the m and b values determined in FIG. 2, calculate refraction values as follows:

r_{B,s,est}=mₛr_{G,s,est}bₛ Spherical Power:

Case 2, Polynomial
Using the p values determined in FIG. 2, calculate refraction values as follows:

r_{B,s,est}=p_{1,s}r²_{G,s,est}+p_{2,s}r_{G,s,est}+p_{3,s} Spherical Power:
For each of the calculated r_{B} values, test if it is between the minimum and maximum values used in determining the p coefficients. If it is not, recalculate that value using the linear method as in Case a.

Case 3, First Order Difference Equation
Using the k values determined in FIG. 2, calculate refraction values as follows:

r_{B,s,est}=k_{1,s}r_{G,s,est}k_{2,s}Δt Spherical Power:

To determine the best estimate spherical refraction for the Bad Eye, let

r_{B,s}=r_{B,s,est}+ΔC_{B}

This procedure is now finished.

I claim:

1. A system allowing the approximate refraction of patients with a central scotoma. The system consists of
a. A set of estimator modules:
i. linear correlation;
ii. polynomial correlation; and
iii. first-order linear difference equation modeling;
each module estimating the following three aspects of an eyeglass prescription:
i. spherical refraction;
ii. cylindrical refraction; and
iii. cylindrical angle;
based on the values of a set of the patient's prescriptions determined before the onset of the central scotoma;
b. Test and Results Comparator Module, which
i. applies the results of the estimator modules to a second set of the patients prescriptions for the Good Eye to estimate the corresponding refractions for the eye with the scotoma;
ii. compares the estimates to the actual refractions in the second set for the eye with the scotoma;
iii. determines which estimation method is best for this patient;
c. Standard Refraction Estimate Module, which
i. takes as input the patient's current refraction for the Good Eye;
ii. applies the estimation method determined in the Tests and Results Comparator Module to be best to refraction of the Good Eye to determine a refraction estimate for the eye with the scotoma
d. Cataract Refraction Estimate Module, which
i. takes as input information on cataracts in each eye, if present;
ii. corrects the spherical refraction in the eye with the scotoma based on progression of refractions in both eyes linked to LOCS III measurements;

2. A method according to claim 1, wherein upon determining that a patient is unable to receive an adequate refraction because of a central scotoma, a vision care provider may
a. Obtain the patients refraction history before the emergence of the scotoma;
b. Enter the refraction history into the Estimator Modules;
c. At each subsequent visit, use the refraction of the patient's Good Eye with the system to determine an estimate of the refraction for the eye with the scotoma;
d. Allow the patient to make a subjective determination of whether the estimated refraction is better than the current eyeglass prescription for the eye with the scotoma.
